(19) United States
(12) Patent Application Publication
(10) Pub. No.: US 2001/0039052 A1
(43) Pub. Date: Nov. 8, 2001

(54) Use of CD34 or a polypeptide derived therefrom as cell-surface/gene transfer marker

(76) Inventor: Axel R. Zander, Ahrensburg (DE)

Correspondence Address: NIXON & VANDERHYE P.C., 8th Floor, 1100 North Glebe Road, Arlington, VA, 22201-4714, US

(21) Appl. No.: 09/836,602

(22) Filed: Apr. 18, 2001

(30) Foreign Application Priority Data
Apr. 18, 2000 (DE) 100 19 075.8

(51) Int. Cl. A61K 48/00; C12N 15/12; C12N 5/08

(52) U.S. Cl. 435/455; 435/320.1; 435/372.3; 424/93.21

(57) ABSTRACT

The present invention concerns the use of CD34 or a polypeptide derived therefrom as cell-surface/gene transfer marker. In particular the object of the invention is a gene transfer vector and host cells transduced by the vector, the vector containing a transgene and a nucleic acid sequence coding for CD34, a fragment of suitable for use in procedures for identification and/or selection of genetically modified cells and in gene therapy. The invention thus further concerns kits for carrying out these procedures and the use of the vector in vitro and as vivo.

The present invention concerns the use of CD34 or a polypeptide derived therefrom as cell-surface/gene transfer marker. In particular the object of the invention is a gene transfer vector and host cells transduced by the vector, the vector containing a transgene and a nucleic acid sequence coding for CD34, a fragment of the same, or a variant of the same. The vector is particularly suitable for use in procedures for identification and/or selection of genetically modified cells and in gene therapy. The invention thus further concerns kits for carrying out these procedures and the use of the vector in vitro and in vivo.

Marker genes are important aids, enabling the identification and selection of genetically modified cells in experimental immunology, haematology and gene therapy. The low affinity nerve growth factor receptor (complete length, LNGFR, or intracytoplasmatically truncated, ΔLNGFR) , variants of human and murine surface antigens such as CD24, CD2, CD4ζ and the enhanced green fluorescent protein such as CD24, CD2, CD4ζ and the enhanced green fluorescent protein these markers however appears to be optimal for clinical practice. A marker suitable for this purpose should be of human origin, to avoid an immune response. Moreover it should only be presented on the genetically modified cells, without being released in the extracellular space. In particular, the markers which can be used for clinical practice should not disturb the physiological functions of the target cells. The surface markers used hitherto are only conditionally suitable, especially for clinical use, i.e. within the framework of gene therapy (cf. B. Fehse et al., Gene Therapy 5 (1998) 429-430).

The task of the present invention is therefore to make available surface markers for the identification and selection of genetically modified cells, which do not have the disadvantages observed in connection with the markers used in the state of the art. The markers should in particular be suitable, within the framework of a gene therapeutic protocol, for the selection/identification of transduced cells of the haematopoietic system, especially primary human or murine T-lymphocytes, without interfering with the haematopoiesis.

According to the invention the task is solved by use of the CD34 surface antigen, a fragment of the same or a variant of the same. In particular the task is solved by the object of the attached claims.

Within the framework of the present invention it was surprisingly ascertained that CD34 is excellently suited for the identification and/or selection of genetically modified cells, with it also being possible to use primary T-lymphocytes as target cells, which after transduction with a gene transfer vector within the framework of a gene therapeutic protocol, can be delivered to a receiver organism, without this resulting in disturbances of the haematopoiesis. Although haematopoietic stem cells express CD34, the haematopoiesis is, unexpectedly, not negatively influenced by the surface marker expressed by the genetically modified cells. In particular it was unexpectedly ascertained that the expression of CD34 on the target cells does not result in a negative influence on cell function and/or cell differentiation. It was moreover possible to show that the expressed surface marker is not toxic to the target cells and—as this is a question of a human protein—it does not promote an immunogenic effect in the recipient.

As CD34 is naturally expressed only on very few cell types, such as human haematopoietic progenitor or stem cells, CD34 is suitable, in a particularly advantageous way, for the purification, enrichment and analysis of cells which do not naturally express CD34, hut especially for the identification and/or selection of genetically modified (transduced) cells, for which technologies exist in the state of the art, and are in particular also permitted for clinical practice, including well characterised monoclonal antibodies, which permit enrichment of the marked cells with a high degree of purity according to GMP (good manufacturing practice) conditions.

For the enrichment and analysis and/or detection of cells which do not naturally express CD34, a nucleic acid sequence coding for CD34 (or for a fragment of the same or a variant of the same) can by means of a vector be introduced into these cells in a form suitable for expression there.

For the marking of genetically modified cells, according to the invention a nucleic acid sequence coding for CD34 (or for a fragment of the same or a variant of the same) is transferred into the target cell together with the gene sequence (transgene) used for the actual transduction. In this case the term transgene is taken to mean a nucleic acid sequence which codes for a protein, polypeptide or peptide, which is expressed in the target cell (or host cell) and confers a new property or function upon this cell. The transgene thus differs from others within the framework of the transduction of transferred nucleic acid sequences in that, the expression product formed in the host cell directly influences the physiological properties and/or the functionality of the cell. With regard to a gene therapeutic application the transgene is a nucleic acid sequence coding for a therapeutically effective protein, polypeptide or peptide.

The object of the present invention is therefore a (gene transfer) vector, which contains

(a) a transgene (optional) and

(b) a nucleic acid sequence coding for a surface marker,

the surface marker being the CD34 surface antigen or a fragment of the same. Also included according to the invention are variants of these sequences, which have the same or essentially identical properties and advantages as to the CD34 surface antigen, including all conceivable variants due to amino acid exchanges, deletions and insertions.

According to a preferred embodiment of the invention, the CD34 surface antigen has the sequence indicated in SEQ ID NO:2, wherein the nucleic acid sequence coding for this protein is preferably the sequence indicated in SEQ ID NO:1. Due to the degeneration of the genetic code, according to the invention variants and mutants of this nucleic acid sequence are included, which code for the same protein. The invention further relates to fragments, mutants and variants of the sequence indicated in SEQ ID NO:1, which code for a protein, polypeptide or peptide comparable with CD34, which has the same or essentially identical properties and is suitable as a surface marker for the identification and/or selection of genetically modified cells.

Within the framework of the present invention, it has been shown that nucleic acid sequences are advantageous, which code for a truncated form of the CD34 surface antigen, i.e. for variants in which proteinase C (PKC) phosphorylation sites are deleted. Also included according to the invention are thus cytoplasmically completely or partially deleted variants of the CD34 surface antigen and gene transfer vectors which contain nucleic acid sequences coding for these variants. This nucleic acid sequence is preferably the sequence indicated in SEQ ID NO:3 or 5.

Within the framework of the present invention, it has been shown that the expression of the truncated/deleted variants of the CD34 protein according to SEQ ID NO:4 and/or 6 allows genetically transduced cells to be detected and selected in an especially suitable way. As the two polypeptides differ from each other only in that the truncated variant (tCD34) is 15 amino acids longer than the deleted variant (dCD34), other variants can naturally also be taken into consideration, whose length lies between the truncated and the deleted variant. Correspondingly the nucleic acid sequence coding for a surface marker will have a length which lies between the lengths of the sequences indicated in SEQ ID NO:3 and 5.

Within the framework of the present invention it has been ascertained that the truncated variant tCD34, compared with the deleted variant, dCD34, has the advantage that the surface antigen is anchored more stably in the membrane of the transduced cells, whereby the identification and selection of the genetically modified cells are clearly improved due to the lower release in the extracellular space. According to a particular embodiment of the invention the nucleic acid sequence coding for the surface marker has in particular the sequence indicated in SEQ ID NO:3 of a sequence derived therefrom by mutation. Due to the degeneration of the genetic code other nucleic acid sequences coding for tCD34 according to SEQ ID NO:4 also naturally come under consideration. Also, nucleic acid sequences which code for variants of the truncated CD34 surface antigen are included (including those obtained by amino acid exchanges, deletions and insertions), which have the same or essential identical properties to the polypeptide indicated in SEQ ID NO:4.

The vector according to the invention can be a non-viral, viral or retroviral vector. A retroviral vector obtained according to a preferred embodiment of the invention, which codes for tCD34, was deposited on 27.03.2000 at the Deutsche Sammlung fur Mikroorganismen und Zellkulturen GmbH (DSMZ), Nascheroder Weg 1b, D-38124 Brunswick (Braunschweig), Germany, under accession No. DSM 13396.

According to a preferred embodiment of the invention the gene transfer vector further contains a nucleic acid sequence coding for a further surface marker such as CD2, EGFP etc., or especially for a therapeutic gene (such as adenosindesaminase (ADA) for healing of the serious immuno-deficiency syndrome ADA-SCID; or others) or a suicide gene. A suicide gene as transgene enables later elimination of transduced cells.

The term “transgene” according to the invention is taken to mean any nucleic acid sequence (foreign gene) which is not naturally contained in the genome of the host or receiver organism or in the vector genome and/or any nucleic acid sequence which is to be transferred to a receiver/host (receiver or host cell).

A further object of the invention is a host cell which is transduced with an aforementioned vector. This host cell is distinct in that in addition to the transgene it also contains the nucleic acid sequence coding for the surface marker and the marker is expressed on the surface of the host cell. The host cell can be a human or non-human (e.g. murine) cell, with (human) T-lymphocytes being preferred.

The invention includes the use of a nucleic acid sequence (marker gene) coding for the CD34 surface antigen or a fragment of the same or a mutant, variant of the same (marker) for the detection of genetically modified cells, in which the nucleic acid sequence is inserted into a gene transfer vector used for genetic modification, which contains a nucleic acid sequence (transgene) to be transferred into the cells, the marker gene and the vector being chosen so that the marker is expressed on the surface of the cells transduced with the vector, the genetically modified cells being identified by specific detection of the marker. The object of the present invention is thus a method to detect genetically modified cells, in which the cells are transduced with a “vector” and the transduced cells are detected by means of selective detection of the marker expressed on the surface of the cells. This detection can be carried out using various methods known to the expert, such as by means of flow cytometric analysis (cf. Fehse et al., Hum. Gene Ther. 8 (1997) 1815-1824) or immunohistochemical methods (Ruggieri et al., Human Gene Ther. 8 (1997) 1611-1623) using monoclonal antibodies.

A further object of the invention is a method for selection of genetically modified cells, in which the cells are transduced with an aforementioned vector and the transduced cells are bound to an agent specific to the surface marker, especially a (monoclonal) antibody, and the cells are thus separated from the genetically non-modified cells (e.g. by magnetic cell sorting, Fehse et al., Hum. Gene Ther. 8 (1997) 1815-1824; or other immuno-adhesion techniques or fluorescence-activated cell sorting, FACS. cf. e.g. Phillips et al., Nat. Med. 2 (1996) 1154-1156).

As already mentioned, the cells used in the method according to the invention are human cells, preferably human T-lymphocytes.

A further object of the invention is a kit to carry out the aforementioned detection method, containing an aforementioned vector, means for the specific detection of the surface marker (including means for carrying out flow cytometry or immunohistochemistry), especially monoclonal antibodies as well as further agents and adjuvants needed to carry out the detection, such as suitable buffers and blocking solutions.

A further object of the intentions is a kit to carry out the aforementioned selection method, containing an aforementioned vector, means for specific binding of the surface marker, such as antibodies coupled to magnetic and/or paramagnetic beads, and further agents and adjuvants necessary to carry out the selection.

As already mentioned above, the vector according to the invention is especially distinguished by its suitability for gene therapeutic applications. The invention therefore further relates to the application of the aforementioned vector for the production of a gene therapeutic drug, especially for the transduction of (human) T-lymphocytes, and a gene therapeutic drug containing this vector. Also included is the use of (human) T-lymphocytes which are transduced with an aforementioned vector, for gene therapeutic treatment. Finally the invention concerns a gene therapeutic drug containing (human) T-lymphocytes which are transduced with the vector according to the invention.

The present invention is described in more detail below with reference to examples, figures and a sequence protocol.

### EXAMPLES

In the following examples the following general techniques are used:

a) Cultivation of primary cells and cell lines.

Mononuclear cells were isolated from the blood of healthy donors by Ficoll gradient centrifugation (Biochrom, Berlin, Germany) (900 g, 20 min.). T-cells were stimulated with 10 ng/ml of OKT-3 (Cilag, Neuss, Deutschland) at a density of 2×10/ml in the presence of 100 U/ml IL-2 (Roche, Mannheim, Germany) in X-Vivo 10 (BioWhittaker, Verviers, Belgium), which contained 8% autologous serum (F. A. Ayuk et al., Gene Ther. 6 (1999) 1788-1792). Jurat and K562 cells were kept in RPMI 1640 which contained 10 fetal calf serum (FCS) and 2 mM glutamine (all from Gibco BRL, Karlsruhe, Germany) . The producer cells of retroviral vectors Phoenix ampho (http://www.standford.edu/group-/holan.NM-phnxr.html, Grignani, F., Kinsella, T., Mencarelli, A. et al., Cancer Res. 58 (1998) 14-19) and PG13 (ATCC CRL-10-56, http://www.ATCC.-org—cf. A. D. Miller et al., J. Virol. 65 (1991) 2220-2224) were kept in Dulbecco's modified Eagle-Medium (DMEM+Glutamax; Gibco BRL), supplemented with 10 heat-inactivated FCS and sodium pyruvate (final concentration, Gibco BRL). All cells were kept at 37° C. in a humidified atmosphere in CO₂ incubators (Heraeus, Hannover, Germany).

b) Gene transfer in K562, Jurkat and primary human T-cells.

Primary T-cells were stimulated with OKT-3 (see above) and cultivated for 3 days in the presence of 100 U/ml IL-2. Jurkat and K562 cells were transduced without prior stimulation. 3 10 cells were suspended in 3 ml filtered Retrovirus-containing supernatant, and 4 μg/ml protamine sulphate (Merck, Darmstadt, Germany) were added. The cells were centrifuged for one hour at 2000 U/min in 6-well-TC plates (Becten Dickinson). Transductions were repeated after 24 hours. The cells were kept in culture for at least 2 days before determination of the gene transfer efficiency.

c) Southern, Northern and Western blot.

Southern, Northern and Western blots were carried out in accordance with standard protocols (F. M. Ausubel et al., Short protocols in molecular biology, 2nd edition, John Wiley and Sons, New York, 1992). Southern and Northern blots were hybridised with radioactively (³³P) marked flCD34. For Western blots and immunoprecipitations cell lysates or cell culture supernatants were used as indicated below.

### Example 1:

Cloning, production and genetic characterisation of flCD34-, tCD34- and dCD34- expressed retroviral vectors

The three types of CD34 which were analysed in this study are represented in FIG. 1a. The cDNAs for flCD34, tCD34 and dCD34 were obtained by means of a RT-PCR with RITA, which had been obtained from human TF1 leukaemia cells, which express CD34 endogenously (FIG. 2b and data from flow cytometry, not shown). In this procedure the RNA from human CD34*-leukaemia cells (TF1) was isolated using the RNeasy Mini Kits (Qiagen, Hilden, German). cDNA was synthesised using an oligo-dT primer and Superscript™ Reverse Transkriptase (Gibco BRL) according to the manufacturer's instructions. The open reading frame for flCD34 was obtained by means of PCR using Pfu polymerase (Stratagene, Amsterdam, Netherlands) and the primers CD34fw 5′-AAGGAAAAAAGCGGCCGCCATGCCGCGGGGCTGGAC-3′ (SEQ ID NO: 7) and CD34rev 5′-TAAGCTTATCACAATTCGGTATCAGCCACCA-3′ (SEQ ID NO: 8). The flCD34 cDNA served as matrix for the production of tCD34 and dCD34 using the primers CD34fw and CD341rev (5′-CAATAAGCTTATCATGGTTCTAGTTCCAGCCTTTCTCCTGTGGGGCT-3′; SEQ ID NO: 9) or CD34fw+CD34srev (5′-CAATAAGCTTATCAATTCATCAGGADATAGCCAG-3′; SEQ ID NO: 10).

The sequencing of the subcloned PCR products revealed an A-G exchange (by comparison with the sequences M81104 and S53811 in GenBank; cf. D. L. Simmons et al., J. Immunol. 148 (1992) 267-271), which leads to an exchange of glutamate for lysin in codon 349 of flCD34. This codon lies in the cytoplasmatic section of the protein and is not present in tCD34 and dCD34, so it was not modified for this study. All variants have the shorter signal peptide which is described in GenBank M81104 (cf. D. S. Simmons et al., J. Immunol. 148 (1992) 267-271). The cDNAs were cloned into the polylinker region of pKS and from the retro-viral expression vector pSFα11 using NotI and HindIII restriction sections. The retroviral vectors (FIG. 1A) use the enhancer/promoter of a variant of the murine spleen focus-forming virus (SFFVp) for initiation of the transcription, which shows moderate activity in murine and human T-cells (C. Baum et al., Virol. 88 (1995) 7541-7547; own results). They also contain an untranslated gag-replacement (GR) leader region which chiefly prevents the expression of aberrant proteins or peptides (M. Hildinger et al., J. Virol. 73 (1999) 4083-4089). Retroviral vectors with high infectious titres were obtained after transduction of the resultant plasmids in Phoenix-ampho packing cells. In this procedure Phoenix cells (amphotropic env-protein) were transduced using the calcium phosphate transduction kit (PeqLab, Erlangen, Germany). In some experiments a plasmid expression vector was co-transduced, which codes for the glycoprotein of the vesicular stomatitis virus (VSV-G), to obtain mixed amphotrope/VSV-G pseudotypes. This led to mixed pseudotypes with titres greater than 10/ml, which allowed the infection of various human and murine cell types. To obtain stable retroviral producer cells and to test the integrity and capacity of the retroviral constructs, retroviral packing cells PG13 and human K562 erythroleukaemia cells were transduced with the supernatants of the transduced Phoenix cells. By subsequent sorting of CD34′-cells and use of MACS technology (magnetic cell sorting; method for enrichment of microbeads-antibody-marked cells by means of special MACS columns which are placed in a strong magnetic field) PG13-producer cell (Gibbon-Ape Leukaemia virus env-proteins) mass cultures could be established. PG13 clones were isolated following limited dilution. Southern-blot analysis of transduced polyclonal PG13 and K562-cells showed the genetic stability of the constructs (FIG. 1b), whereby it was ensured that the following analyses were carried out with cells, in which the transgenes were correctly processed. Supernatants of the cells were collected after six hours' incubation at 37° C. in X-Vivo 10.

(F. A. Ayuk et al., Gene Ther. 6 1999) 1788-1792). Viral titres were determined by transduction of Jurkat cells with serial dilutions of virus-containing supernatants and subsequent FACS analysis (B. Fehse et al., Hum. Gene Ther. 8 (1997) 1815-1824).

### Example 2

Enrichment of genetically modified cells by CD34

The flow cytometry showed that in murine fibroblast PG13 cells and in human K562 haematopcietic cells, the retroviral vectors expressed all three CD34 variants (flCD34, tCD34 and dCD34) each in large quantities. The differences in transfer-efficiencies correspond to the titres of the packaging cells. Polyclonal populations of PG13 and K562 cells, which expressed the three different versions of CD34 could easily be enriched to a high purity using cell sorting based on immuno-affinity (MACS technology) (FIGS. 2A, B). To do so, the cells were enriched three days after transduction using the CD34 progenitor cell isolation kit (Miltenyi Biotec, Bergisch-Gladbach, Germany) in accordance with the manufacturer's instructions (B. Fehse et al., Hum. Gene Ther. 8 (1997) 1815-1824). After enrichment the cells were marked with phycoerythrin-coupled anti-CD34 (HPCA-2) not interfering with the antibodies used for enrichment. The new phenotype remained stable in culture for several months both for K562 (FIG. 2B) and also for PG13 (data not shown). No influence of the variants on the proliferation of the transduced cells could be detected. However flow cytometry showed that the cell surface expression of dC34 was weaker than that of the other two variants.

### Example 3:

The residual cytoplasmatic part of tCD34 is involved in the membrane anchoring of the cell surface molecule.

To examine the mechanisms on which the observed expression differences are based, the three transduced variants of CD34 were examined at the level of the transcript and of the protein. This took place using polyclonal populations of K562 and PG13 cells, which had been transduced with the three versions of the retroviral CD34 vectors and which had been immuno-selected for expression of CD34. The histogram of cell surface marking with CD34, with which the different variants were compared, demonstrated that the expression of dCD34 was an order of magnitude lower than that of tCD34, which was just as strongly represented on the cell surface as flCD34 (FIG. 3A). Comparable data could be achieved with human Jurkat lymphocytes (not shown,. Northern blot analysis (FIG. 3B) confirmed that the three variants had different transcript lengths and identical total expression rates. The stronger signal from flCD34 in K562 cells could be explained by a higher loading with RNA, which was revealed by methylene-blue staining of the membrane before hybridisation (not shown). This result shows that the 3′ end of the CD34 cDNA contains no sequences that influence the processing of the RNA. The loss of expression must therefore originate from differences in the processing of the CD34 protein (FIGS. 3C, D).

Western blot analyses of the cell lysates confirmed the results obtained by flow cytometry. FIG. 3C shows the weaker expression of dCD34, compared with the two naturally occurring forms tCD34 and flCD34. This argues against the unlikely possibility that dCD34 is retained in the cytoplasma. The Western blot showed that dCD34, like tCD34, has a reduced molecular weight compared with flCD34.

In summary these results show that the membrane anchoring of dCD34, which lacks the cytoplasmatic part, but still contains the complete transmembrane-domain, can be unstable. To investigate this question, non-concentrated cell culture supernatants of K562 cells which had been transduced either with dCD34 or tCD34 (FIG. 3D) were immuno-precipitated. In fact CD34 was detected in clearly increased quantities in the supernatants of the dCD34 expressing cells. Therefore the reduced cell surface expression of dCD34 can be explained by release from the membrane. From this it can be concluded that the residual cytoplasmatic part of the tCD34 has an important function in membrane anchoring and through this presents the release of the cell surface protein.

### Example 4

Expression of tCD34 in human T-lymphocytes

On the basis of the results available, tCD34 was selected as the most interesting variant for the marking of cell surfaces and the immuno-selection of genetically modified cells. An essential application of this technology, is the enrichment of genetically modified lymphocytes for use in adoptive immunotherapy in patients (C. Bonini et al., Science, 276 (1997) 1719-1724; P. Tiberghien et al., Hum. Gene Ther. 8 (1997) 615-624. It was therefore examined, whether the retroviral vector-mediated expression of tCD34 in human T-calls is feasible.

The transduction of human T-cells is best carried out using retroviral vectors which are pseudo-standardized with the env-protein of the Gibbon Ape Leukaemia Virus (GALV) . These vectors can be produced in PG13 cells (F. A. Ayuk et al., Gene Ther. 6 (1999) 1788-1792; B. A. Bunnell et al., Blood 89 (1997) 1987-1995). Stable clones of PG13 cells, which express the retroviral vector Sfα11tCD34 at high titres were obtained by limited dilution of the corresponding mass culture. On 27.03.2000 a specimen of this vector was deposited with the DSMZ in Brunswick (see above) under no. DSM 13396. Supernatants of these producer cells were used for the gene transfer in human Jurkat T-lymphoblastoma cells (FIG. 4A) and in primary peripheral blood lymphocytes (PBLs) which had been stimulated with IL-2 and OKT-3 (FIG. 4B) . As was determined by flow cytometry, the expression of tCD34 in Jurkat cells was slightly higher than in primary T-cells. A similar difference was also observed with a retroviral vector which contains identical transcription control elements, but expresses EGFP instead of tCD34 (not shown). The expression of tCD34 was strong enough to enable a separation of these cells using MACS technology both with Jurkat and PBLs. The immuno-selected cells were always obtained with high purity (FIG. 4). These cells were observed in culture for up to a week. They remained CD34 positive and showed no obvious changes in proliferation or morphology.

### Example 5:

The use of tCD34 to track genetically modified murine erythroid myeloid and lymphoid cells in vivo.

Finally it was examined whether tCD34 can be used to mark retrovirally modified murine haematopoietic cells, including primary T-cells in vivo, which are obtained after transplantation with multi-potent precursor cells. Non-fractionated mononuclear gene marrow cells were transduced with retroviral vectors which express tCD34 or as a control EGFP. These vectors were identical with reward to the cis-acting elements, which control the gene expression (FIG. 1). In particular the cells were obtained as follows: bone marrow cells were obtained from the fibia and the femurs of male C57B1/6J donor mice (age 12-16 weeks) 4 days after intraperitoneal delivery of 5-fluorouracil (Sigma) (150 mg/kg) . Mononuclear cells were prestimulated in IMDM medium (Iscove's Modified Dulbecco's Medium), which had been supplemented with 20 fetal calf serum, glutamate, 100 U/ml penicillin, 100 μg/ml streptomycin and a normal growth factor cocktail containing murine IL-3 (10 ng/ml), human IL-6 (200 U/ml) and murine SCF (50 ng/ml). Recombinant growth factors were obtained from Strathmann Biotech (Hannover, Germany). After two days prestimulation cell-free supernatants of mixed ampho/VSV-pseudo-standardized retroviral particles were added. The multiplicity of infection (MOI) amounted to 0.7 infectious particles per cell, calculated from previous titration of aliquots of supernatants on SC-1 fibroblasts. Polybrene (sigma) (4 μg/ml) was added and the cells were centrifuged for an hour at 2000 U/min. This process was repeated three times within 48 hours, with a pause of at least 8 hours between the individual transduction steps. Comparable transduction rates were achieved, by using cell-free supernatants with equivalent titres. One day after the transduction as completed, the cells were transplanted into the tail veins of lethally irradiated (10 Gy) female recipients (n=6 for each vector) at a dose of 2.2×10 cells per mouse. Nine weeks after the transplantation, the peripheral blood cells were analysed by means of flow cytometry with respect to the expression of the transgene in erythroidal cells (determined by scatter properties), and in myeloid cells, B-lymphocytes and T-lymphocytes (identified using the monoclonal antibodies CD11b, B220, and a combination of CD4 and CD8). Cells of these lines, including T-lymphocytes, expressed tCD34 in easily detectable quantities (FIG. 5A) , from which it can be concluded that the transgene and surface-marked cells normally differentiate in vivo, and transgene expression remains stable. Whilst EGFP and tCD34 could be found in a comparable frequency in myeloid and erythroid cells, it was possible to observe a tendency for lymphoid cells with tCD34 to become somewhat less marked (FIG. 5B).

### LEGENDS TO FIGURES

FIG. 1

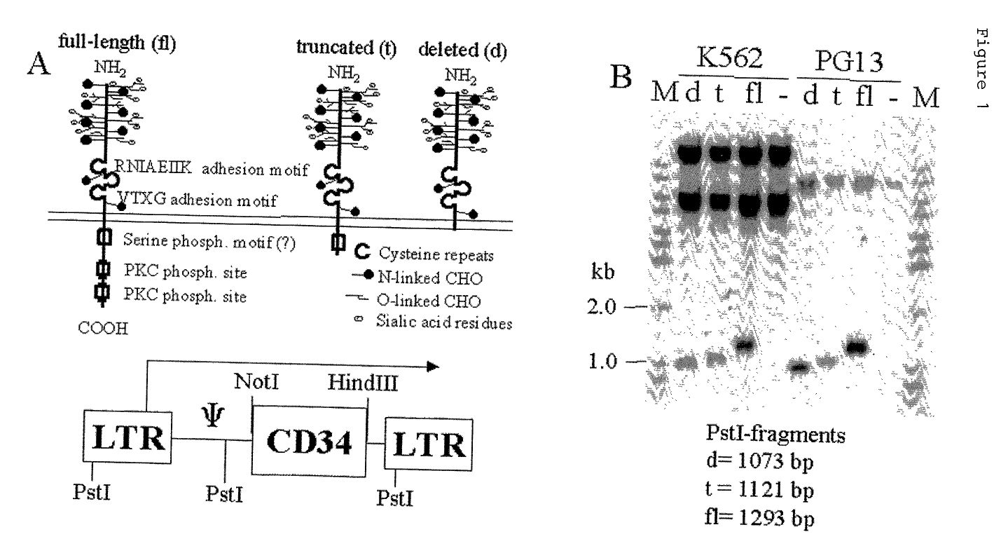

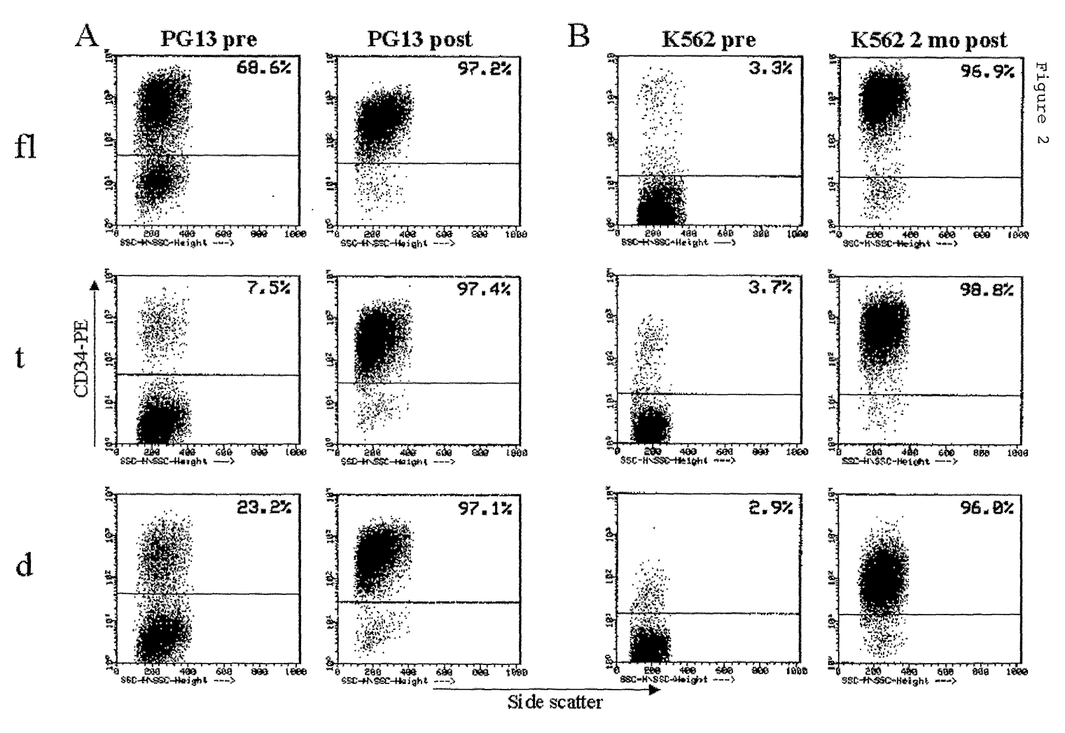

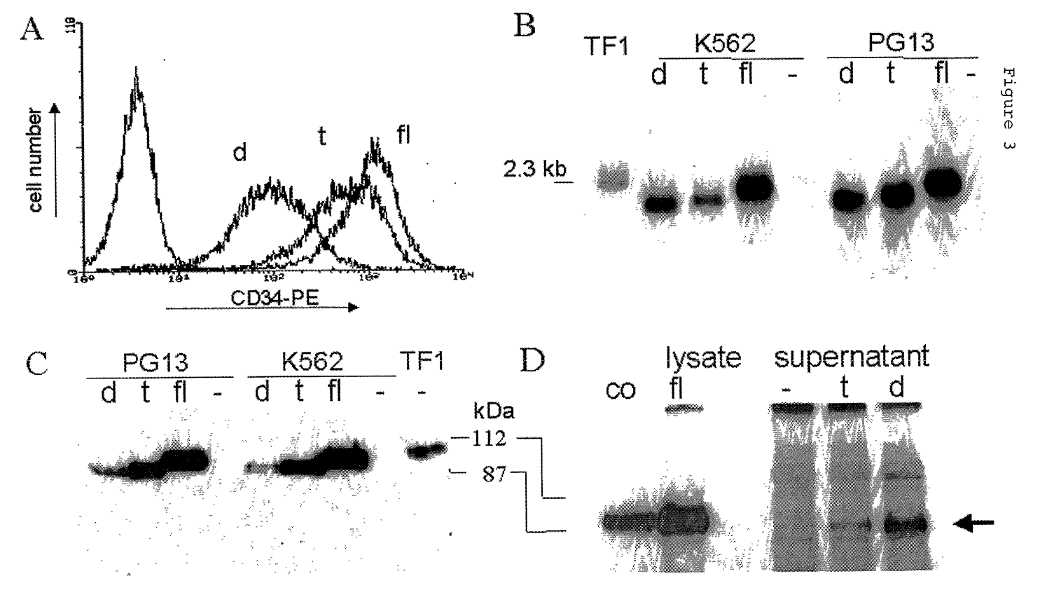

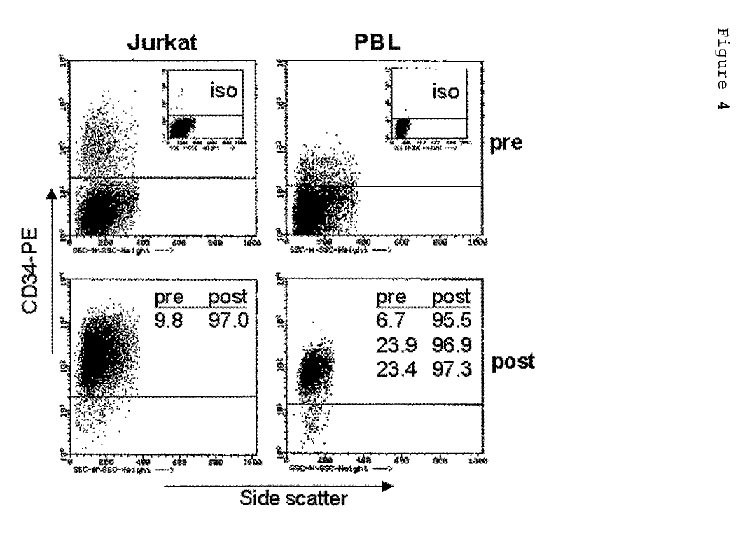

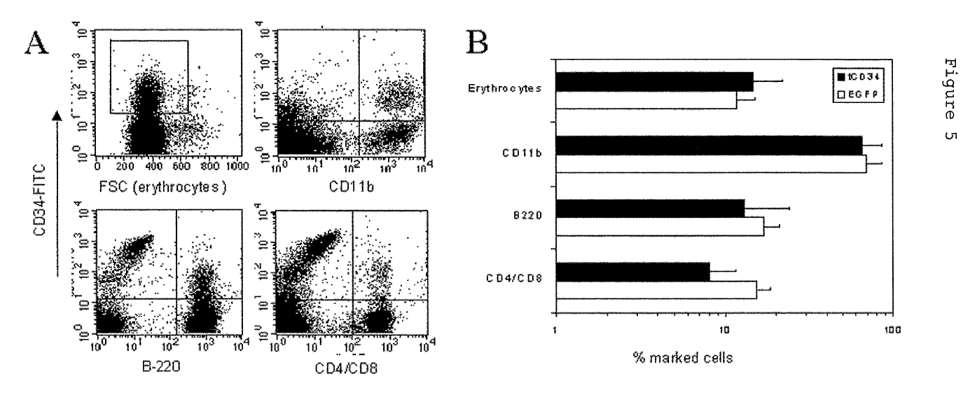

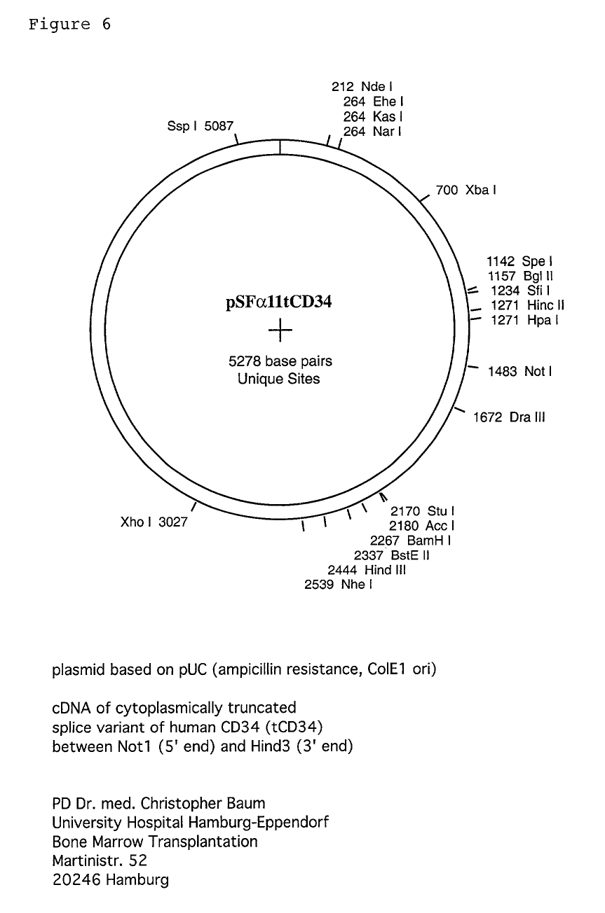

Retroviral vectors for the expression of all three variants of CD34.

(A) (above) Schematic representation of the three CD34 variants, (modified from D. S. Krause et al., Blood 87 (1996) 1-13): The cytoplasmatic tails of flCD34, tCD34 and dCD34 comprise 73, 16 and 1 amino acids; (bottom) proviral form of the retroviral vectors used for expression of CD34. Size of the proviral form is approx. 2.6 kb. The long terminal repeat (LTR) is from a variant of the murine spleen focus forming virus. The untranslated leader region contains the retroviral packing signal (ψ) without gag sequences. The cDNAs were inserted using NotI and HindIII restriction sites.

(B) Southern blot analysis of the immuno-selected K562 and PG13 cells transduced with the three different retroviral CD34 expression vectors. Genomic DNAs were digested with PstI. Hybridization with the probe human flCD34 revealed correct insert lengths, as indicated. Hybridisation signals of higher molecular weight result from cellular genes. M, DNA molecular weight marker ladder mix WBI Fermentas, St. Leon-Rot, Germany).

FIG. 2

Enrichment of PG13 and K562 cells based on stable expression of retrovirally transduced CD34.

(A) PG13 cells after transduction with Phoenix supernatants before (PG13 pre, analysed two days after transduction) and immediately after (PG13 post) enrichment using immuno-affinity columns.

(B) K562 cells before (K562 pre, analysed two days after transduction) and two months after (K582 2 mo post) enrichment using immuno-affinity columns.

FIG. 3

The cell surface expression of dCD34 is reduced because of release into the cellular supernatant.

(A) Overlay histogram of CD34 expression in uncloned, K562 cells transduced with dCD34 (d), tCD34 (t) or flCD34 (fl); determination by flow cytometry two months after immunoaffinity-based enrichment with a resulting purity of 96%, 98% and 97% respectively cell surface expression of dCD34 is about one order of magnitude reduced as compared with tCD34 and flCD34. Similar results were obtained with Jurkat cells (not shown).

(B) Northern blot analysis of total RNA, harvested from mass cultures of K562 and PG13 cells transduced with the three variants of the CD34 expression vectors or untransduced cells (−). TFI cells are shown as a positive control, with an endogenous expression of flCD34 (lower band, approx. 2.3 kb) and tCD34 (upper band, approx. 2.5 kb) (Krause et al., loc. cit., 1996). Comparison with the loading control (methylene-blue stained filter, not shown) confirmed comparable expression levels of all three retroviral RNAs in transduced cells.

(C) Western blot analysis of cell lysates, harvested from transduced and untransduced PG13 and K562 cells (the same cultures as in (A) and (B)). Weaker expression of dCD34 is confirmed. It should be noted that dCD34 and tCD34 have a lower molecular weight (approx. 100 kDa), compared with flCD34 (approx. 110 kDa). 20 μg protein were loaded per lane, CD34 was detected using the monoclonal antibody QBEND 10, HRP conjugated goat-anti-mouse IgG and the SuperSignal™ West Pico Chemoluminescence substrate (Pierce, Rockford, Ill.

(D) Immuno-precipitation with HPCA-2 antibodies from cellular supernatants of K562 cells, which had been transduced with tCD34 (t) or dCD34 (d) or from non-transduced cells (−). The arrow indicates soluble CD34. Cell lysate of K562:flCD34 cells are shown as a positive control (co), just as immuno-precipitated lysates of K562:flCD34 cells. Detection was carried out by means of Western blot, as described above.

FIG. 4

Enrichment of genetically modified human T-cells, including primary peripheral blood lymphocytes (PBL) using retroviral vectors, which expressed tCD34. The isotype control is shown as an insert (iso) in the dot blot analysis, which was obtained from the (pre) magnetic cell sorting (MACS technology). In independent experiments the purity after enrichment (post) was 95.5, 96.9 and 97.3 for PBL after an initially positive signal of 6.7, 23.9 and 23.4.

FIG. 5

Expression of tCD34 on murine peripheral blood cells in vivo.

(A) Representative dot blots which show the expression of tCD34 in the blood of mice, status 9 weeks after bone marrow transplantation with retrovirally marked cells. Peripheral blood cells of C57B1/6J mice were obtained by bleeding the tail veins and were tested by means of flow cytometry for expression of tCD34. By means of scatter profile and derivation-specific antibodies, myeloid cells (CD11b), B-cells (B220), T-cells (cocktail of CD4 and CD8) and erythrocytes were differentiated, the latter being determined by size corresponding to the forward scatter (ESC). The markers were adjusted on the basis of isotype controls.

(B) The marking efficiency with tCD34 is comparable with results obtained with EGFP. The multiplicity of the infection was adapted to an equal gene transfer efficiency, as indicated by equal marking in myeloid and erythroid cells. It should be noted that there is a tendency for lymphocytes with tCD34 to be slightly less marked than those with EGFP. The average values are shown (percentage of marker-positive cells) with standard deviations. There were six animals within each experimental group.

FIG. 6

pUC-based plasmid (Ampicillin-resistance, ColE1 ori). The cDNA of the cytoplasmatically truncated variant of human CD34 (tCD34) is located between NotI (5′-end) and HindIII (3′-end).

In the plasmid pSFalpha11tCD34 the reading frame of tCD34, a splice variant of the human CD34 differentiation antigen, is located between NotI and HindIII. It lies functionally under the control of an eukaryontic promoter with a subsequent 5′-untranslated region (Region 150 bp upstream of XbaI to NotI, comprising sequences of the murine retroviruses MPSV and MESV). The corresponding signal in the long terminal repeat (LTR) of the SFFV retrovirus initiates polyadenylation; this LTR is located between HindIII and XhoI. The transcription-regulating signals are only recognized in the case of transfection in eukaryontic cells. The sequences downstream from XhoI to ca. 150 bp upstream from XbaI comprise the plasmid backbone based in pUC19, which mediates the ampicillin resistance in transformed bacteria and bears the replication origin for the plasmid.

1. Gene transfer vector which contains
a) a transgene and
b) a nucleic acid sequence coding for a surface marker,
characterized in that the surface marker is the CD34 surface antigen or a fragment of the same or a variant of the same.

2. Vector according to claim 1, characterized in that the nucleic acid sequence codes for a surface marker in accordance with SEQ ID NO: 2, 4 or 6 or for a fragment or a variant of the same.

3. Vector according to claim 1 or 2, characterized in that the nucleic acid sequence codes for the surface marker is the sequence indicated in SEQ ID NO: 1, 3 or 5 or for a fragment, a mutant or variant of the same.

4. Vector according to claims 1 to 3, characterized in that it is a retroviral vector.

5. Vector according to claims 1 to 4, characterized in that it contains a nucleic acid sequence coding for a further surface marker.

6. Vector with the accession no. DSM 13396.

7. Vector characterized in that it contains a nucleic acid sequence coding for the amino acid sequence according to SEQ ID NO: 6, a fragment or a variant of the same.

8. Vector according to claim 7, characterized in that it contains the nucleic acid sequence according to SEQ ID NO: 5, a fragment, a mutant or a variant of the same.

9. Host cell, characterized in that it is transduced with a vector according to claims 1 to 8.

10. Host cell according to claim 9, characterized in that it is a human cell.

11. Host cell according to claim 10, characterized in that it is a T-lymphocyte.

12. Method for the detection of genetically modified cells, characterized in that the cells are transduced with a vector according to claims 1 to 5 and the transduced cells are identified by detection of the surface marker.

13. Method for the selection of genetically modified cells, characterized in that the cells are transduced with a vector according to claims 1 to 5, bound to an agent specific to the surface marker, and separated from the genetically unmodified cells.

14. Method for the detection and analysis of cells, characterized in that the cells are transduced with a vector which contains a nucleic acid sequence coding for the surface marker CD34, a fragment of the same or a variant of the same, and the transduced cells are identified by detection of the surface marker, in which the cells do not naturally express CD34, a fragment or a variant of the same.

15. Method for enriching cells which do not naturally express CD34, a fragment or a variant of the same, characterized in that the cells are transduced with a vector which contains a nucleic acid sequence coding for the surface marker CD34, a fragment of the same, or a variant of the same, and the transduced cells are bound to an agent specific to the surface marker, and separated from the cells which do not express the surface marker.

16. Method according to claim 14 or 15, characterized in that the nucleic acid sequence codes for a surface marker according to SEQ ID NO: 2, 4 or 6 or for a fragment or a variant of the same.

17. Method according to claims 14 or 15, characterized in that the nucleic acid sequence coding for the surface marker is the sequence indicated in SEQ ID NO: 1, 3 or 5 or a fragment, mutant or variant of the same.

18. Method according to claims 14 to 17, characterized in that the vector is a retroviral vector.

19. Method according to claims 14 to 20, characterized in that the vector corresponding to DSM 13396 is used.

20. Method according to claims 12 to 19, characterized in that the cells are human cells.

21. Method according to claims 20, characterized in that the cells are T-lymphocytes.


SEQUENCE LISTING (10 sequences)

SEQ ID NO: 1 - DNA, 1122 bp, Homo sapiens
  CDS (1)..(1122): CD34 (complete length)
atgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcaggaaaacgggaccggccaggccacctccagaaacggccattcagcaagacaacacgtggtggctgataccgaattgtga

SEQ ID NO: 2 - protein, 373 aa, Homo sapiens
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 3 - DNA, 951 bp, Homo sapiens
  CDS (1)..(951): CD34 (truncated variant)
atgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggaactagaaccatga

SEQ ID NO: 4 - protein, 316 aa, Homo sapiens
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 5 - DNA, 906 bp, Homo sapiens
  CDS (1)..(906): CD34 (deleted variant)
atgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaattga

SEQ ID NO: 6 - protein, 301 aa, Homo sapiens
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMN

SEQ ID NO: 7 - DNA, 36 bp, Artificial sequence
  Description of the artificial sequence Primer CD34fw
aaggaaaaaagcggccgccatgccgcggggctggac

SEQ ID NO: 8 - DNA, 31 bp, Artificial sequence
  Description of the artificial sequence Primer CD34rev
taagcttatcacaattcggtatcagccacca

SEQ ID NO: 9 - DNA, 47 bp, Artificial sequence
  Description of the artificial sequence Primer CD34lrev
caataagcttatcatggttctagttccagcctttctcctgtggggct

SEQ ID NO: 10 - DNA, 34 bp, Artificial sequence
  Description of the artificial sequence Primer CD34srev
caataagcttatcaattcatcaggaaatagccag